(19) United States
(12) Patent Application Publication
(10) Pub. No.: US 2012/0330128 A1
(43) Pub. Date: Dec. 27, 2012

(54) PET-MRI CONVERGENCE SYSTEM

(75) Inventors: Hyun-Wook Park, Daejeon (KR); Myung-Sung Song, Daejeon (KR)

(73) Assignee: KOREA ADVANCED INSTITUTE OF SCIENCE AND TECHNOLOGY, Daejeon (KR)

(21) Appl. No.: 13/530,638

(22) Filed: Jun. 22, 2012

(30) Foreign Application Priority Data
Jun. 27, 2011 (KR) 10-2011-0062100

(51) Int. Cl. A61B 5/055; A61B 6/02

(52) U.S. Cl. 600/411

(57) ABSTRACT

A Positron Emission Tomography (PET)-Magnetic Resonance Imaging (MRI) convergence system may be provided that includes: a magnet bore having a cylindrical shape, an outer wall and an inner wall; a gradient coil disposed to contact with the inner wall of the magnet bore; a body coil which is disposed to contact with the inner wall of the gradient coil, emits an RF pulse signal, and has a first through-hole formed therein; and an MRI RF receiving coil detecting MRI data corresponding to the RF pulse signal. The PET detector includes a second through-hole formed therein and contacts with one end of the body coil such that the first through-hole is connected to the second through-hole. The examinee transporter is transportable through the first through-hole and the second through-hole.

### CROSS-REFERENCE TO RELATED APPLICATION

This application claims priority under 35 U.S.C. §119(a) from Republic of Korea Patent Application No. 10-2011-0062100 filed on Jun. 27, 2011, which is incorporated by reference herein in its entirety.

### BACKGROUND

1. Field of the Invention

The present invention relates to a positron emission tomography (PET)-magnetic resonance imaging (MRI) convergence system.

2. Description of the Related Art

In general, a positron emission tomography (hereafter, referred to as PET) device is used to form an image of a particular human organ or a tumor or used to form an image for diagnosing biochemical phenomena of metabolic activity areas. The PET device generates a tomographic image by marking a radioactive isotope which emits positrons on various basic metabolites and injecting it to a human body, and by detecting, outside the body, gamma ray generated from the interaction between the positron and the metabolites.

A PET image obtained by the PET device generally has low resolution. For this reason, in the past, the low resolution of the PET image has been compensated by using the PET device together with a computer tomography (hereafter, referred to as CT) device.

However, a CT image obtained by the CT device generally has a low contrast to soft-tissues. Therefore, in place of the CT device, a positron emission tomography-magnetic resonance imaging (PET-MRI) convergence system has been developed which has a high contrast to soft-tissues of a human body, provides molecular images and functional images and uses the PET device together with a magnetic resonance imaging (MRI) device without radiation exposure.

FIG. 1 shows a conventional PET-MRI system. The conventional PET-MRI system 100 has a structure obtained by stacking a PET detector and an MRI RF coil. The PET detector and the MRI RF coil have the same field of view (FOV) (see Performance Test of an LSO-APP Detector in a 7-T MRI Scanner for simultaneous PET/MRI, Pichler et al, JNM, Vol. 47, No. 4, April 2006).

In the conventional PET-MRI system, the PET detector is RF shielded and placed outside the MRI body coil in order to reduce the influence of a high frequency signal of the MRI on the PET. An RF shielding conductor of the PET detector deteriorates the performance of the MRI coil and generates an eddy current of a gradient field to degrade an MRI image. Further, since the PET detector is disposed outside the MRI body coil, a gamma ray emitted from a patient is attenuated and scattered by the MRI body coil, so that the size of the signal can be reduced. The element of the PET detector which is used in an MRI magnet is made of a non-magnetic material. However, elements including a capacitor or a resistance element of a pre-amplifier are difficult to be made of the non-magnetic material. Residual magnetism present in the elements may affect the main magnetic field uniformity of the MRI. The RF shield structure of the PET detector and electronic circuits generate the eddy current by interacting with the signals generated from the gradient coil (a gradient field generator) of the MRI. The eddy current reduces the signal-to-noise ratio of the MRI image.

In the prior PET-MRI system, both of the PET device and the MRI device are necessary to generate either only a PET image or only an MRI image. As a result, the efficiency of the system is degraded and it is difficult to repair the elements of the PET system or equipments related to the PET system due to the strong magnetic field of the MRI.

### SUMMARY

One embodiment is a Positron Emission Tomography (PET)-Magnetic Resonance Imaging (MRI) convergence system. The PET-MRI convergence system includes an MRI system, a PET detector and an examinee transporter. The MRI system includes: a magnet bore having a cylindrical shape, an outer wall and an inner wall; a gradient coil disposed to contact with the inner wall of the magnet bore; a body coil which is disposed to contact with the inner wall of the gradient coil, emits an RF pulse signal, and has a first through-hole formed therein; and an MRI RF receiving coil detecting MRI data corresponding to the RF pulse signal. The PET detector includes a second through-hole formed therein and contacts with one end of the body coil such that the first through-hole is connected to the second through-hole. The examinee transporter is transportable through the first through-hole and the second through-hole.

The PET detector is attachable to and removable from the PET-MRI convergence system.

The PET-MRI convergence system performs a PET scan and an MRI scan of an examinee in a pipeline manner.

In the PET-MRI convergence system, the PET detector further includes an RF shield which surrounds the PET detector and shields the RF pulse signal emitted from the MRI RF coil.

In the PET-MRI convergence system, the PET detector further includes a data processor which receives PET data from a PET detecting unit and processes the data. The data processor is separated from the MRI system.

Another embodiment is a PET-MRI convergence system. The PET-MRI convergence system includes an MRI system, a PET detector and an examinee transporter. The MRI system includes: a magnet bore having a cylindrical shape, an outer wall and an inner wall; a gradient coil disposed to contact with the inner wall of the magnet bore; a body coil which is disposed to contact with the inner wall of the gradient coil, emits an RF pulse signal, and has a first through-hole formed therein; and an MRI RF receiving coil detecting MRI data corresponding to the RF pulse signal. The PET detector includes a second through-hole formed therein and contacts with one end of the body coil such that the first through-hole is connected to the second through-hole. The examinee transporter is transportable through the first through-hole and the second through-hole. The PET-MRI convergence system further includes: an RF shield which is attachable to and removable from the PET-MRI convergence system and surrounds the PET detector and shields the RF pulse signal emitted from the MRI RF coil; and a data processor which receives PET data from a PET detecting unit of the PET detector and processes the data. The data processor is separated from the MRI system.

### BRIEF DESCRIPTION OF THE DRAWINGS

FIG. 1 shows a cross section of a conventional PET-MRI convergence system;

FIG. 2 shows a cross section of a PET-MRI convergence system 200 according to an embodiment of the present invention;

FIG. 3a shows a cross section of an MRI system of the PET-MRI convergence system shown in FIG. 2;

FIG. 3b is a cross sectional view of the PET detector shown in FIG. 2b as viewed from a side of the PET detector;

FIG. 3c is a cross sectional view of the PET detector shown in FIG. 2b as viewed from a top of the PET detector;

FIGS. 4a, 4b and 4c are views for describing the operation of the PET-MRI convergence system 200 according to the embodiment of the present invention;

FIG. 5a shows the operation of the separated MRI system according to the embodiment of the present invention; and

FIG. 5b shows the operation of the separated PET detector according to the embodiment of the present invention.

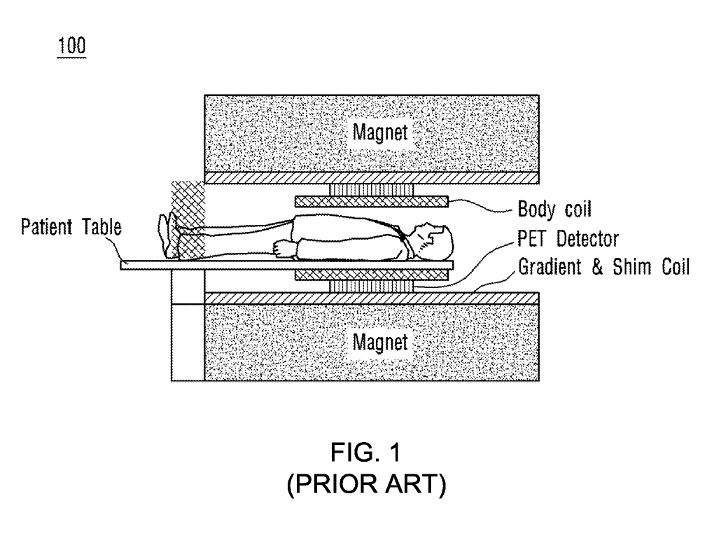

### DETAILED DESCRIPTION

The Figures (FIG.) and the following description relate to preferred embodiments of the present invention by way of illustration only. It should be noted that from the following discussion, alternative embodiments of the structures and methods disclosed herein will be readily recognized as viable alternatives that may be employed without departing from the principles of the claimed invention.

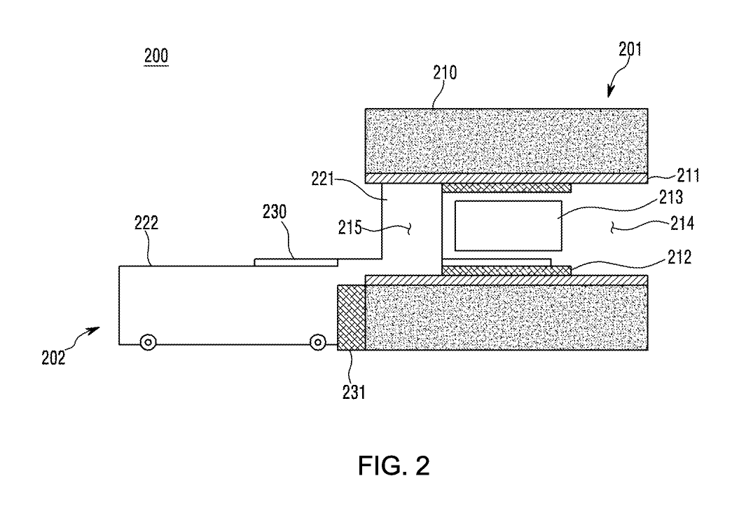

FIG. 2 is a cross section of a PET-MRI convergence image acquisition device according to an embodiment of the present invention.

As shown in FIG. 2, the PET-MRI convergence system 200 according to the embodiment of the present invention includes an MRI system 201 for MRI scans and a PET detector 202 for PET scans.

Referring to FIG. 2, a PET-MRI convergence system 200 may include a magnet bore 210, a gradient coil 211 forming a gradient magnetic field, a body coil 212 transmitting an RF pulse signal to an examinee, an MRI RF receiving coil 213 receiving MRI data corresponding to the RF pulse signal, a first through-hole 214 and a second through-hole 215 in which the examinee can be received for PET-MRI scans, a PET detecting unit 221 detecting a PET signal emitted from the examinee, a transporter 230 transporting the examinee, an RF shield 222 shielding the RF signal emitted from the MRI system, and a transporter driver 231 controlling the movement of the transporter 230.

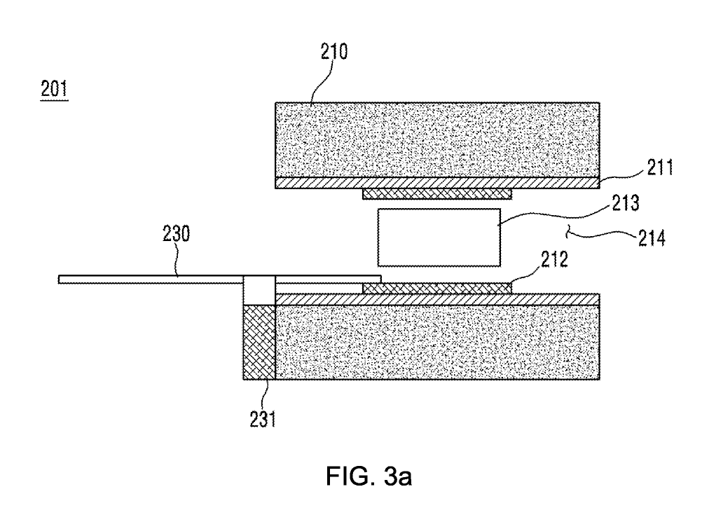

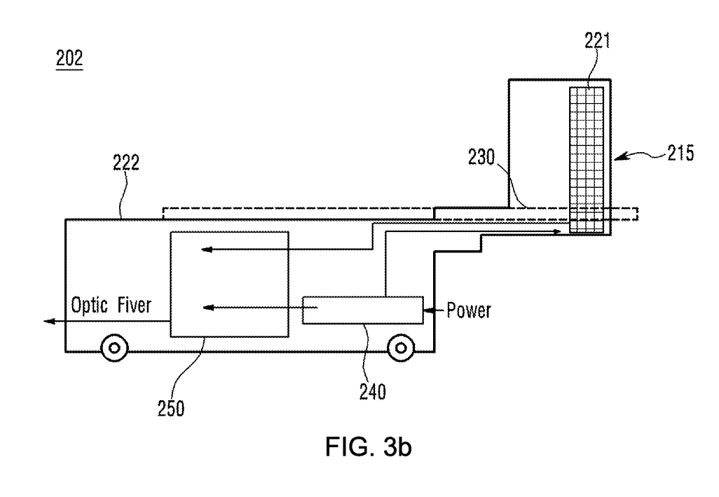

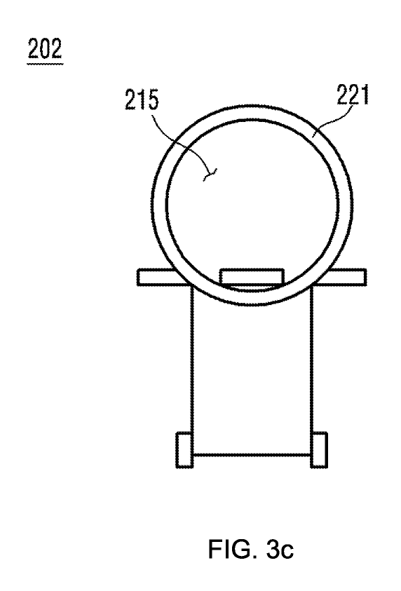

FIGS. 3a, 3b and 3c show the MRI system and the PET detector respectively, each of which has been separated from the PET-MRI convergence system 200 of FIG. 2.

Referring to FIG. 3a, the MRI system 201 may include the magnet bore 210 forming a static magnetic field, the gradient coil 211 forming the gradient magnetic field, the body coil 212 transmitting an RF pulse signal to the examinee, the MRI RF receiving coil 213 receiving the RF signal from the examinee, the first through-hole 214 through which the examinee is transported by the transporter 230, and the transporter driver 231 controlling the movement of the transporter 230.

The magnet bore 210 is a main magnet and means a structure which surrounds the movement axis of the transporter 230. Here, it is recommended that the magnet bore 210 should have a cylindrical structure comprised of an outer wall and an inner wall. The body coil 212 is disposed adjacent to the inner wall of the gradient coil 211. When the static magnetic field and the gradient magnetic field are generated from the magnet bore 210 and the gradient coil 211 respectively, the body coil 212 has a signal transmission function of emitting an RF pulse signal corresponding to Lamor frequency. The MRI RF receiving coil 213 has a receiving function of receiving MRI data emitted from the examinee.

When the magnet bore 210 operates, a static magnetic field parallel with the movement axis of the transporter 230 is generated within the first through-hole 214 by the magnet bore 210.

Here, the operation of the body coil 212 will be described in detail. When the RF pulse signal is emitted from the body coil 212, the emitted RF pulse signal is added to the examinee on the transporter 230. Then, the examinee emits the RMI data corresponding to Lamor frequency of the added RF pulse signal. At this moment, the MRI RF receiving coil 213 collects the MRI data emitted from the examinee.

The PET-MRI convergence system 200 according to the embodiment of the present invention includes the first through-hole 214 allowing the transporter 230 transporting the examinee to easily come in and out. The first through-hole 214 is formed within the body coil 212.

Referring to FIG. 3b, the PET detector 202 includes the PET detecting unit 221 detecting a PET signal emitted from the examinee, the second through-hole 215 through which the examinee is transported by the transporter 230, the RF shield 222 shielding the RF signal emitted from the MRI system, a power distributor 240 supplying electric power to the PET detector 202, and a data processor 250 processing the data detected by the PET detecting unit 221.

The PET detector 202 may be independently formed in an attachable and removable manner in the PET-MRI convergence system 200. When a PET-MRI convergence image is required, the PET detector 202 may, as shown in FIG. 2, operate adjacent to the MRI system.

When the examinee enters a PET imaging region, the PET detecting unit 221 detects a PET data (a gamma ray) emitted from the examinee. The PET detecting unit 221 is located on one end of the PET detector 202 and may contact with the side of the body coil 212.

The transporter 230 transports the examinee along the movement axis, i.e., the central axes of the PET detecting unit 221, the first through-hole 214 and the second through-hole 215. The transporter 230 does not include a receiver receiving the MRI data or the PET data. For this reason, the transporter 230 has a simple structure and makes it possible to prevent the examinee from feeling inconvenience and uneasiness due to an enclosed space. Further, since the PET imaging region is separated from the MRI imaging region, a sufficient space allowing the patient to be placed is obtainable.

The RF shield 222 is disposed to cover the entire outer wall of the PET detector 202 and shields the RF pulse signal emitted from the body coil 212.

The power distributor 240 distributes electrical power to the PET detector 202.

The data processor 250 collects the PET data obtained from the PET detecting unit 221 and generates the PET image.

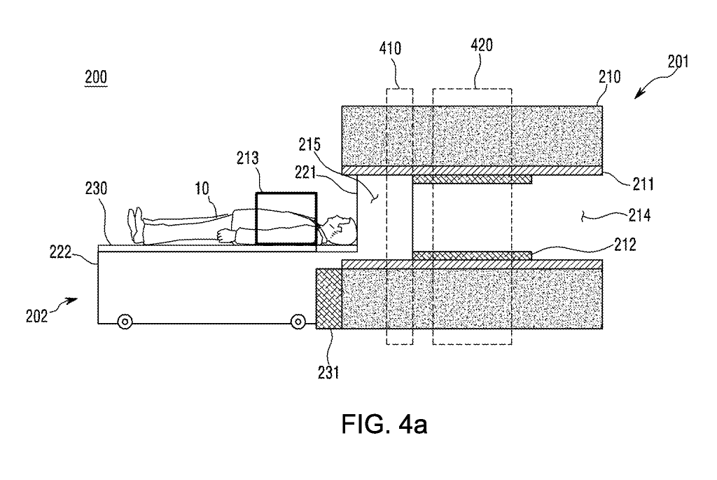

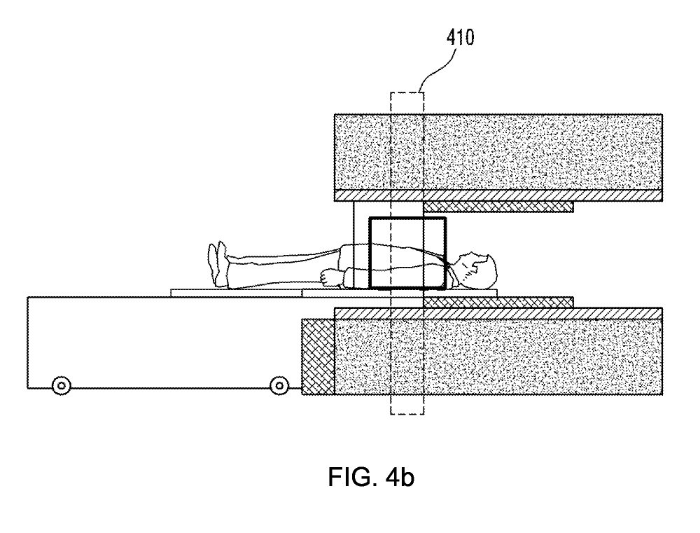

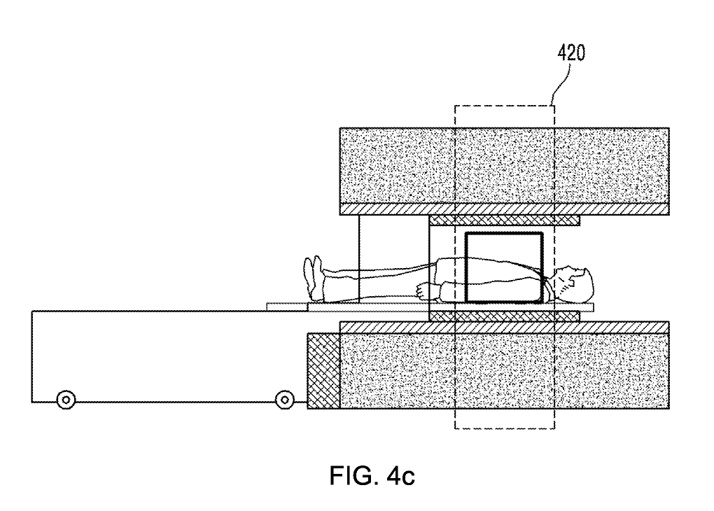

FIGS. 4a, 4b and 4c are views for describing the operation of the PET-MRI convergence system according to the embodiment of the present invention.

FIG. 4a shows a state before the examinee 10 is scanned by the PET-MRI convergence system 200.

Referring to FIG. 4a, the PET-MRI convergence system 200 includes an MRI imaging region 420 for obtaining a magnetic resonance data of the examinee 10 and a PET imaging region 410 for obtaining a positron emission tomography data of the examinee 10.

Referring to FIG. 4b, in detecting the PET data, the transporter 230 is moved in such a manner that a body part to be scanned of the examinee 10 (for example, abdomen) is placed in the center of the PET imaging region 410, and then the PET data is detected. When the abdomen of the examinee 10 is placed in the Pet imaging region, the head of the examinee may be placed in the MRI imaging region 420, thereby detecting the MRI data of the head as well as the PET data of the abdomen.

Referring to FIG. 4c, subsequently, the transporter 230 transports the abdomen of the examinee 10 to the MRI imaging region 420, and then the MRI data of the abdomen is detected. Here, not only the PET data of the head of the examinee 10 but also the PET data of the lower part of the body of the examinee 10 can be detected at the same time in the Pet imaging region 410.

As described above, the PET-MRI convergence system 200 according to the exemplary embodiment of the present invention is able to detect the MRI data of the examinee 10 together with the PET data of the examinee 10 as well, or is able to drive the operations of the PET and MRI at a predetermined time interval.

The PET-MRI convergence system 200 according to the exemplary embodiment shown in FIGS. 2 to 4 of the present invention is able to simultaneously detect the MRI data and the PET data of a predetermined body part or the whole body of the examinee 10. Particularly, the body coil 212 within the MRI imaging region is fixed and mounted on the center of the magnet bore 210, so that the MRI data for the whole body of the examinee 10 can be easily obtained.

The PET imaging region 410 and the MRI imaging region 420 are arranged in series in the direction of the movement axis of the transporter 230 transporting the examinee. Therefore, the PET imaging region 410 and the MRI imaging region 420 are formed in the form of a pipeline, so that the examinee can be easily scanned and a time required for taking a scan of the whole body of the examinee can be reduced.

Further, when the PET data of the examinee 10 is detected, the PET detecting unit 221 is RF shielded so that a structure for detecting the MRI data is not provided around the examinee 10. Accordingly, pure PET data of the examinee 10 can be obtained without artifacts.

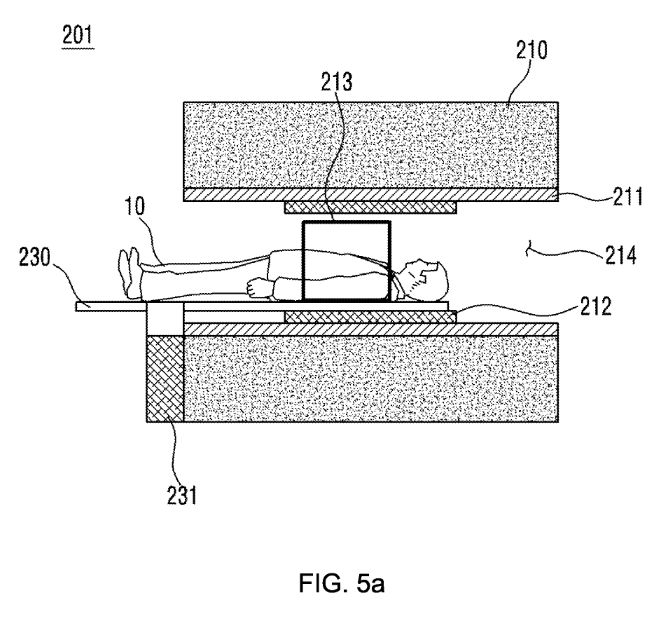

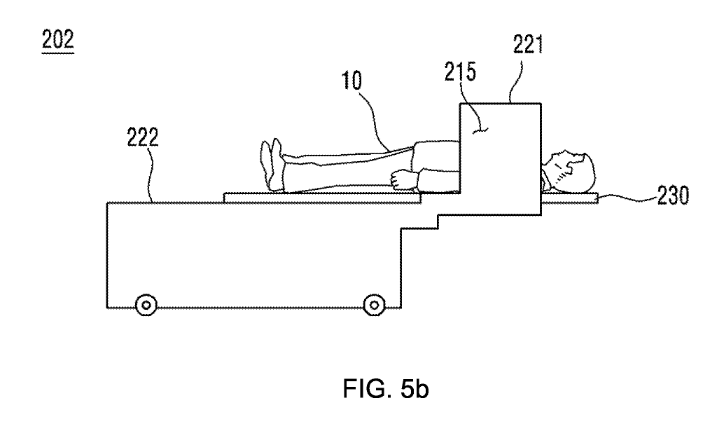

FIGS. 5a and 5b show that the MRI system and the PET system are separated from each other and independently operated respectively.

As shown in FIGS. 5a and 5b, the PET-MRI convergence system according to the embodiment of the present invention can be used by being separated into the MRI system and the PET system when it is desired to generate either the PET image or the MRI image. As a result, it is possible to variously use the PET-MRI convergence system and to improve the efficiency of the scanning process.

Through the present invention, it is possible to overcome the limitation of the field of view (FOV) of the prior PET-MRI system, to have a PET-MRI system structure including a field of view (FOV) making it possible to scan the whole body of an examinee, and to reduce problems such as a high magnetic field, high frequency high power interference and low frequency high power interference, magnetism distortion, signal-to-noise ratio reduction and Eddy current or the like. The PET-MRI system according to the embodiment of the present invention is able to maximize a space for MRI scan. Thus, MRI data distortion caused by the PET, artifacts and signal-to-noise ratio reduction can be decreased and a PET-MRI image quality can be improved by reducing the negative effects of the PET data due to the MRI.

The features, structures and effects and the like described in the embodiments are included in at least one embodiment of the present invention and are not necessarily limited to one embodiment. Furthermore, the features, structures, effects and the like provided in each embodiment can be combined or modified in other embodiments by those skilled in the art to which the embodiments belong. Therefore, contents related to the combination and modification should be construed to be included in the scope of the present invention.

Although embodiments of the present invention were described above, these are just examples and do not limit the present invention. Further, the present invention may be changed and modified in various ways, without departing from the essential features of the present invention, by those skilled in the art. That is, the components described in detail in the embodiments of the present invention may be modified. Further, differences due to the modification and application should be construed as being included in the scope and spirit of the present invention, which is described in the accompanying claims.

What is claimed is:

1. A Positron Emission Tomography (PET)-Magnetic Resonance Imaging (MRI) convergence system comprising an MRI system, a PET detector and an examinee transporter,
wherein the MRI system includes:
a magnet bore having a cylindrical shape, an outer wall and an inner wall;
a gradient coil disposed to contact with the inner wall of the magnet bore;
a body coil which is disposed to contact with the inner wall of the gradient coil, emits an RF pulse signal, and has a first through-hole formed therein; and
an MRI RF receiving coil detecting MRI data corresponding to the RF pulse signal,
wherein the PET detector includes a second through-hole formed therein and contacts with one end of the body coil such that the first through-hole is connected to the second through-hole,
and wherein the examinee transporter is transportable through the first through-hole and the second through-hole.

2. The PET-MRI convergence system of claim 1, wherein the PET detector is attachable to and removable from the PET-MRI convergence system.

3. The PET-MRI convergence system of claim 1, wherein the PET-MRI convergence system performs a PET scan and an MRI scan of an examinee in a pipeline manner.

4. The PET-MRI convergence system of claim 1, wherein the PET detector further comprises an RF shield which surrounds the PET detector and shields the RF pulse signal emitted from the MRI RF coil.

5. The PET-MRI convergence system of claim 1, wherein the PET detector further comprises a data processor which receives PET data from a PET detecting unit and processes the data, and wherein the data processor is separated from the MRI system.

6. A Positron Emission Tomography (PET)-Magnetic Resonance Imaging (MRI) convergence system comprising an MRI system, a PET detector and an examinee transporter,
wherein the MRI system includes:
a magnet bore having a cylindrical shape, an outer wall and an inner wall;
a gradient coil disposed to contact with the inner wall of the magnet bore;
a body coil which is disposed to contact with the inner wall of the gradient coil, emits an RF pulse signal, and has a first through-hole formed therein; and
an MRI RF receiving coil detecting MRI data corresponding to the RF pulse signal,
wherein the PET detector includes a second through-hole formed therein and contacts with one end of the body coil such that the first through-hole is connected to the second through-hole,
wherein the examinee transporter is transportable through the first through-hole and the second through-hole,
and wherein the PET-MRI convergence system further includes:
an RF shield which is attachable to and removable from the PET-MRI convergence system and surrounds the PET detector and shields the RF pulse signal emitted from the MRI RF coil; and
a data processor which receives PET data from a PET detecting unit of the PET detector and processes the data, wherein the data processor is separated from the MRI system.
